(19) United States
(12) Patent Application Publication
(10) Pub. No.: US 2008/0281088 A1
(43) Pub. Date: Nov. 13, 2008

(54) Method And Device For The Isolation Of Nucleic Acid From Nucleic Acid-Comprising Material

(76) Inventors: Rolf Andre Mank, Rhenen (NL); Martin Jaap Zevenbergen, Ede (Gld) (NL)

Correspondence Address: BROWDY AND NEIMARK, P.L.L.C.;624 NINTH STREET, NW, SUITE 300, WASHINGTON, DC, 20001-5303, US

(21) Appl. No.: 11/587,137

(22) PCT Filed: May 26, 2005
(86) PCT No.: PCT/NL2005/000387

§ 371 Date: Sep. 10, 2007

(30) Foreign Application Priority Data
May 26, 2004 (NL) 1026271

(51) Int. Cl. C07H 1/06

(52) U.S. Cl. 536/25.4

(57) ABSTRACT

Method for the isolation of nucleic acid from nucleic acid comprising material, comprising the following steps: a) provision of a container suitable for the isolation of nucleic acid; b) introducing the nucleic acid comprising material and a desiccant into the container; c) drying the nucleic acid comprising material in the container; d) subjecting the nucleic acid comprising material in the container to a method for the isolation of nucleic acid from the nucleic acid comprising material; e) isolation of nucleic acid.

### FIELD OF THE INVENTION

The present invention relates to a method and a device for the isolation of nucleic acid from nucleic acid comprising material. In particular the invention relates to a method and a device for the combined collection and drying of nucleic acid comprising material and the isolation of nucleic acid of improved quality from the nucleic acid comprising material.

### STATE OF THE ART

The collection of nucleic acid comprising material and the isolation of nucleic acid from nucleic acid comprising material, and in particular DNA from DNA comprising material, are frequently the first steps that are taken in the process of studying DNA, for example for genetic analysis such as the identification of genes, markers or SNPs (Single Nucleotide Polymorphisms). The conventional isolation of DNA is one of the tried and tested methods in the state of the art and has been amply described in the text books, such as in Sambrook & Russel (2001) “Molecular Cloning: A Laboratory Manual” (3^{rd} edition), Cold Spring Harbor Laboratory, Cold Spring Harbor Laboratory Press. Rapid and efficient methods for the isolation of DNA from rice have also been described in Williams et al., Nucleic Acids Research, 1994, (22), 1917-1918, and Zheng et al., Rice Genetics, Newsletter, 1995, (12), 255-258.

The isolation of DNA with characteristics that make it suitable for a wide variety of analyses and in particular for restriction analysis, cloning and selective sequence amplification with the aid of the Polymerase Chain Reaction (PCR) is routinely possible starting from virtually all species in all stages of development, in particular plants, if the material is fresh, is stored cryogenically (deep-frozen) or is freeze-dried. Suitable DNA is usually characterised by the fact that it is not degraded or is degraded to only a limited extent (visible as a ‘smear’ on electrophoresis gel), is not contaminated or is contaminated to a limited extend with other (foreign) DNA and/or contains no proteins and/or secondary plant substances or contains these to a limited extent.

However a disadvantage in the art is that many samples have to be collected at locations that are far removed from the location where the eventual DNA isolation and further analysis will take place. One of the solutions available in the art is the cooled transport of the DNA comprising sample, for example on dry ice (solid carbon dioxide). Although this solution is adequate for transport over short distances, problems arise when transport over longer distances is necessary or during transport from locations where adequate cryogenic facilities are not available.

It is known to dry DNA comprising material, such as plant material, on silica (Chase et al., Taxon, 1991, (40), 215-220) or calcium sulphate (Drierite®) (Liston et al., Ann. Missouri Bot. Garden, 1990, 77, 859-863). For this purpose 4 to 6 gram plant material is placed as a whole or in smaller pieces of approximately 2 cm² in size into a bag together with 50-60 g silica and is dried for one or two days. The surplus silica is then removed (and optionally re-used). DNA isolation takes place in the conventional manner by grinding each sample containing DNA-comprising material in liquid nitrogen at −197° C. This is a method that according to the authors works well if it is a few leaf samples that are concerned, but for the routine isolation of DNA from a large diversity of samples of different origin this is a time-consuming method.

Many of the known techniques, such as, inter alia, those described above by way of illustration, result in DNA of lower quality than is required or desired for some analytical methods, such as the AFLP technique (Vos et al., Nucleic Acids Research 1995 (23) 4407-4414). Moreover, these methods are associated with the disadvantage that for the isolation of DNA from a multiplicity of samples the techniques used to date are associated with high risks of cross-contamination. These are clear disadvantages of the known techniques.

The inventors have set themselves the aim of avoiding or reducing the disadvantages mentioned in this application and also set themselves the aim of providing a rapid and efficient method for the collection and drying of nucleic acid or material containing DNA and which enables the isolation of nucleic acid or DNA from the nucleic acid or DNA comprising material, which method does not make use of cryogenic conditions. The inventors also set themselves the aim of providing a method that is able to supply DNA of high quality (DNA with a high molecular weight) that is suitable for use in analyses such as the AFLP technique. The inventors also set themselves the aim of providing a device for the combined collection and drying of DNA-comprising material, which device is also suitable for the isolation of DNA from the DNA-comprising material.

### BRIEF DESCRIPTION OF THE INVENTION

Surprisingly it has now been found that by introducing the nucleic acid or DNA-comprising material into a container in which a desiccant is already present or to which a desiccant is added subsequently or at the same time, then allowing the nucleic acid or DNA-comprising material to dry by the action of the desiccant and subjecting the nucleic acid or DNA-comprising material in the container to a method for the isolation of nucleic acid or DNA, preferably in the presence of the desiccant present, nucleic acid or DNA of exceptional quality can be isolated.

In a first embodiment the invention therefore relates to a method for the isolation of nucleic acid from nucleic acid comprising material, comprising the following steps:
a) provision of a container suitable for the isolation of nucleic acid;
b) introducing the nucleic acid comprising material and a desiccant into the container;
c) drying the nucleic acid comprising material in the container;
d) subjecting the nucleic acid comprising material in the container to a method for the isolation of nucleic acid from the nucleic acid comprising material;
e) isolation of the nucleic acid.

### DETAILED DESCRIPTION OF THE INVENTION AND PREFERRED EMBODIMENTS

The advantages of the invention lie in the advantageous combination where in a first step the nucleic acid comprising material is introduced directly into the container from which the nucleic acid will eventually be isolated. In a preferred embodiment the nucleic acid comprising material is DNA, but other types of nucleic acids can also be used in the method. (For the sake of simplicity, the term DNA is used where nucleic acid can be read. This is without prejudice to the fact that the invention is also suitable for nucleic acids other than DNA, such as RNA, MRNA, etc.) The step in which the DNA-comprising material is introduced directly into the container from which the DNA is isolated avoids the transfer of DNA-comprising material from the one container to the other container during the complete procedure from the collection of DNA-comprising material to isolation, whereby cross-contamination and loss of DNA can occur. This reduction in the number of operations is less important for single samples but becomes of significant importance (including economically) when larger numbers, preferably more than, for example, a few tens, hundreds or thousands, of samples have to processed. The reduction of the number of operations is also of less importance when analysing PCR products by means of sequencing, because any contamination that has arisen can be discovered, but other techniques, which are also highly customary, such as RFLP, RAPD and AFLP, do not detect this contamination. It is therefore also advantageous if a method is available that is able to preclude such a risk systematically.

The DNA-comprising material can be of plant, animal or human origin and can be in solid or liquid form, such as originating from blood, sperm, mucus or muscle tissue. In principle the method is suitable for any form of DNA-comprising material. The method is preferably suitable for plant material, but can also be used for, for example, insects and fms of fish.

The DNA-comprising material can be collected and introduced into the container in a manner known per se, for example manually or with the aid of tweezers. For plant material, and in particular leaf material, a leaf punch provided with a punch container is very suitable. Such a leaf punch is available from Rabbit Tool USA Inc., Rock Island Ill. USA (http://www.rabbittool.com). Manual methods are very suitable, such as using a cork borer or an apple corer. In the majority of plants 15-30 mg leaf material corresponds to approximately 1.5 cm² leaf surface area, which corresponds to approximately 5 punches (or disks) with a diameter of 6 mm (cork borer) or 1 punch with an apple borer.

In a preferred embodiment either the desiccant is already present in the container or is introduced into the container a short period before or is preferably added to the container at the same time as, immediately or after a short period after the collection of the DNA-comprising material. In this context short period means a period that is short enough to prevent degradation of DNA-comprising material, for example by the action of the enzymes, etc. that are still present and active in the DNA-comprising material. The advantage of adding the desiccant shortly before or shortly after the collection of the DNA-comprising material lies in the fact that in such cases the desiccant can be stored in a separate, properly moisture-tight container, which in view of the inherently hygroscopic nature of the desiccant is an advantage and contributes to uniform quality of drying and comparability of the samples. Here it is equally the case that this is also advantageous for a few samples (up to about ten), but the advantage becomes exceptional when tens to thousands and more samples are concerned.

In principle the container can be any container that is suitable for the collection of DNA-comprising material and the subsequent isolation of DNA therefrom. Examples of such containers are, preferably closable, test tubes, small pots and the like. So-called Micronics Tubes (Micronics BV Lelystad, The Netherlands) http://www.micronic.com) are particularly suitable.

The step involving drying the material by means of the desiccant in the container serves, as is known per se, to preserve the material by extracting water. This is a step that advantageously can take place, for example, during transport from the location where the DNA is collected to the location where the DNA is isolated. Compared with the cryogenic methods, such as transport in the presence of dry ice, this manner of drying during transport at room temperature is advantageous and appreciably less vulnerable. Compared with the known method of Chase et al. that is based on the use of silica, where contamination can arise by drying the material in a very large excess of silica in the first instance and then re-using the silica, in the case of the present invention, in contrast, contamination by drying the material in the container can be avoided.

In one embodiment the desiccant or the combination of desiccants is selected from the group of desiccants that are inert with respect to DNA. In a preferred embodiment one or more desiccants are selected from the group consisting of silica, alumina, aluminium silicates (Molsieve®), calcium sulphates (Drierite®), magnesium aluminium silicates, porous clay materials, diatomaceous earth, zeolites, salts of (poly)acrylic acid and mixtures and compositions of the said materials. Silica, which may or may not be combined with a moisture indicator, is preferred as desiccant. In principle, any form of silica that is known in the art as a suitable desiccant can be used in the present invention. Taking the tips described in this application into account, a person skilled in the art can easily determine which size and type of silica is most appropriate for the case in question.

In general silica can absorb approximately 31% of its own weight of water (compare calcium sulphate 10-14%). The weight ratio of silica to DNA-comprising material is thus preferably at least 3, that is to say there is at least a 3-fold weight excess of silica compared with the DNA-comprising material in the container. This weight ratio can also be lower, depending on the moisture content of the DNA-comprising material, and this falls within the invention. A higher weight ratio is preferably more than 4, more preferably more than 5 and most preferably more than 6. This has the advantage that the desiccant does not become saturated too rapidly and the drying process can proceed quickly and efficiently. Comparable considerations in respect of the ratio of desiccant to DNA-comprising material and the moisture content thereof apply for the other desiccants mentioned. The weight ratio of the desiccant in general to DNA-comprising material and the moisture content thereof is so chosen that the amount of water that can be extracted from the DNA-comprising material is at least such that the processes in the cell and in particular the processes in the cell that contribute to the degradation of DNA cease or are retarded to such an extent that these processes no longer have an adverse effect on the quality of the DNA isolated.

In a preferred embodiment the DNA-comprising material is dried under non-cryogenic conditions. In a preferred embodiment the DNA-comprising material is dried at a temperature between 0 and 50° C., preferably between 10 and 40° C., more preferentially between 15 and 30° C. and most preferentially between 18 and 25° C. Drying at ambient temperature is most preferred.

Depending on the desiccant used the DNA-comprising material is dried for at least one hour, preferably at least two hours and more preferably at least three or four hours. Drying for at least 8 hours, preferably at least 10 hours, is preferred. In a preferred embodiment drying is carried out for at least one day, preferably at least two days, more preferably at least three days. The minimum drying period is sufficiently long for the processes in the cell and in particular the processes in the cell that contribute to degradation of DNA to cease or to be retarded such that these processes no longer have an adverse effect on the quality of the DNA isolated. The plant material dried in accordance with the invention is characterised by excellent preservation of the DNA material present, which is manifested in the exceptionally long shelf life of DNA dried and stored in this way, of more than a few months up to a few years, without the quality of the DNA eventually isolated deteriorating significantly.

The DNA-comprising material in the container is subjected to a method for the isolation of DNA, preferably in the presence of the desiccant for at least part, that is to say at least one step, preferably two or more, of the method for the isolation of the DNA. Said method preferably comprises the size reduction or grinding of the DNA-comprising material in the container, extraction of DNA from the DNA-comprising material in the container and separation of the DNA from the DNA-comprising material in the container.

In one embodiment the desiccant is present in the container during the size reduction or grinding of the plant DNA-comprising plant material in the container. The DNA-comprising material can be crushed or ground in the container in any known manner. For example, crushing using tweezers with a blunt end or a curved pipette point. These methods are mainly suitable for a few to at most a few tens of samples. For large numbers of samples, however, manual mechanical grinding is a limiting step (Csaikl et al., Plant Mol. Biol. Rep. (1998), 16, 69-86). It is therefore advantageous to make use of grinding techniques such as, for example, the ‘Mixer Mill’ available via Bratt technologies LLc, East Orange, N.J., USA and other techniques which are based on the addition of grinding balls or beads. In the present invention the grinding beads are added to the container containing the desiccant and the DNA-comprising material and are then subjected to a size reduction treatment. For instance, a set of grinding beads can be added to each tube in a Micronics® rack containing 96 tubes, after which the DNA-comprising material is ground by vigorous agitation of the rack with (closed) tubes. The extraction and the isolation can take place simultaneously in a comparable manner in all 96 tubes.

In a further embodiment the desiccant is present in the container during the extraction of DNA from the DNA-comprising material in the container. The extraction of the DNA is carried out in accordance with protocols known per se, such as are described in Sambrook et al. (see above), or as described in Drabkova et al. Plant Molecular Biology Reporter (2002), 20, 161-175. The extracted DNA in the extraction buffer is separated from the other plant material and the desiccant and transferred to a second container from which the DNA is isolated by means of methods known per se.

It has been found that the DNA isolated in this way is of good quality as described above and is suitable for the generation of genetic fingerprints, for example using techniques such as the AFLP technique. It has also been found that in the case of plants such as strawberries, which in conventional methods for DNA isolation show substantial contamination with secondary plant substances such as polyphenols and polysaccharides, DNA of an excellent quality is obtained, where contamination with said secondary plant substances is essentially absent or substantially reduced. The power of the method lies in the combination of the various operations of the method in one container, which results in DNA of good yield and quality. Furthermore, the method is characterised by the avoidance of possibly contaminating operations.

In a further aspect the invention is characterised by a device which comprises a closable container, which container is suitable for carrying out the method. The device is characterised by a closable container which is suitable for the collection of DNA-comprising material and contains a desiccant and which container is also suitable for the isolation of DNA in the presence of the desiccant.

The invention will now be described in more detail on the basis of the following examples.

### EXAMPLES

### Materials and Methods

Use is made of a set of Micronics Tubes:
1. Reusable tube holder 96 with cover and ID label 96; NL and non-US product code M225-00 or USA product code BD352110;
2. Reusable tube holder with unprinted tubes NL and non-US product code M225-96 or USA product code BD352112;
3. PPN tubes NL and non-US product code M226-C2 or USA product code BD352115;
4. Cover strip 8, non-sterile (2×100 in number) NL and non-US product code M227-05 or USA product code BD352118;
5. Cover strip 8 bulk (1000 in number) NL and non-US product code M227-C2 or USA product code BD352122;
6. Re-usable tube holder filled with printed tubes, NL and non-US product code M225-RP or USA product code BD352111;

The 1.4 ml Micronics tubes are filled ¼ (approx. 0.25 gram) or ½ (approx. 0.5 gram) with Sigma S-4883, 28-200 mesh, mixed with silica gel with a humidity indicator (Merck 1.01925≈1.3 mm) and four different plants (pepper, lettuce, cucumber and tomato). For each combination six tubes were filled with silica before and six tubes after harvesting leaf material. Five leaf punches (leaf disks) were harvested from each plant. The tubes containing silica gel and leaf disks were dried for 4 days at room temperature after which DNA was isolated without removing the silica gel, in accordance with the following protocol:
Two small stainless steel beads were added to the tube comprising leaf material and silica gel.
Grind the plant material in the mixer mill by shaking this for 30 sec. at the maximum setting.
Add 500 μl CTAB extraction buffer e.g. with the aid of the 8-channel pipette.
Grind the plant material again in the mixer mill by shaking this for 30 sec at the maximum setting.
Check that the material has been ground well; repeat if necessary.
Remove the tubes from the Micronic holder and place these in a water bath at 60° C. for 60 min.
Then allow the tubes to cool to room temperature on ice.
Add 250 μl chloroform/isoamyl alcohol (24:1) e.g. with the aid of the 8-channel pipette and mix the tubes for approximately 5 min. on the rocker platform.
Separate the phases by centrifuging these for 10-20 min at 3,500 rpm.
Take a new holder with new tubes and put 200 μl isopropanol into the tubes.
Pipette 200 μl of the aqueous phase into the isopropanol. Cover the whole with the 8-strip caps and mix the whole briefly.
Pellet the DNA by centrifuging for 15 min at 3,500 rpm.
Remove the caps from the tubes and stick a tube holder on the box and decant the liquid.
Allow the pellet to dry.
Dissolve the DNA in 100 μl T₁₀E₁.
   Composition of CTAB buffer per litre: 100 ml TRIS pH 7.5; 140 ml 5M NaCl; 20 ml 0.5M EDTA pH 8.0; 740 ml Milli Q.

This experiment was repeated with maize using tubes ¼ filled with silica gel. The material was dried for three days at room temperature, after which the DNA was isolated in the presence of the silica in accordance with the above protocol.

A standard AFLP procedure (Vos et al., see elsewhere) was carried out on the above isolated DNA from all tubes and genetic fingerprints were generated. Fresh material from the same plants was used as control material and freeze-dried in the normal manner.

It was difficult, but not impossible, to isolate sufficient supernatant ftom the tubes that were ½ filled with silica for DNA precipitation after chloroform extraction. The yield and the quality of the DNA originating from tubes that were ¼ filled with silica were visually checked on 1% agarose gel and found to be good. No difference was found between tubes that were filled with silica before or after placing the leaf material in the tubes. DNA isolation from maize also proceeded satisfactorily; the yield and quality were somewhat lower than in the case of the other plants, but adequate for a good analysis.

In general the quality of the genetic fingerprints by means of AFLP was good, both on the basis of a visual inspection and on the basis of the scoring of markers compared to the freeze-dried control material.

In a second experiment over a period of two weeks, 6 plants (radish, tomato, melon, cucumber, lettuce, strawberry, maize and pepper) were tested 3 times per week in the manner described above to see whether storing the material on silica gel for a longer period would lead to differences in the eventual fingerprints. Once again the control material was fresh freeze-dried leaf material. DNA yields were good and the AFLP fingerprints were of constant quality based on visual inspection and on the basis of marker scores.

In a third experiment 3 Micronics racks with tubes filled with silica gel were sent by post. After a few weeks the sealed packs containing the racks filled with leaf samples were returned by post. DNA was isolated in the manner described above and the AFLP fingerprints were likewise of good quality based on visual inspection and based on marker score.

In a fourth experiment the method was tested on blood and other material.

For animals without DNA comprising erythrocytes (mammals, birds) 0.3 ml (300 microlitres) whole blood is introduced into 1.4 ml Micronics tubes. For animals with DNA comprising erythrocytes (fish, reptiles) 0.01 ml (10 microlitres) whole blood is introduced into 1.4 ml Micronics tubes.

Typical protocols which are suitable for the isolation of DNA from DNA-comprising material that has been collected and dried on silica can vary somewhat from source to source. Mammalian blood and bird blood are subjected, in the tube and in the presence of the silica, to a conventional lysis with NH4CL buffer (8.3 g/l NH4CL, 1.0 g/l KHCO3, 3.72 mg/l Na2EDTA), suspension in DNA extraction buffer (400 mM NaCl, 10 mM Tris HCL pH 8.0, 0.2 mM Na2EDTA), treatment with proteinase K buffer (50 mM TRis-HCL pH 8.0, 10 mM EDTA, 0.4 mg/ml Prot-K) and cold ethanol precipitation. Fish blood and sperm are preferably treated correspondingly with proteinase K buffer and a standard chloroform/phenol protocol. For tissue, such as skin or muscle tissue, the protocol for mammalian blood was followed, but with an amount of material (and thus a ratio with the silica present in the container) that corresponds to that which was used for leaf material, approx. 15-30 mg.

In all cases sufficient DNA was obtained which was of adequate quality for carrying out AFLP.

1. A method for of isolating nucleic acid from nucleic acid-comprising material in a container, comprising the steps of:
(a) introducing nucleic acid-comprising material into a container suitable for nucleic acid isolation;
(b) before, during or after step (a), adding a desiccant to the container;
(c) drying the material in the container; and
(d) isolating, within the container, the nucleic acid from the material;
thereby isolating the nucleic acid in the container.

2. A method according to claim 1, wherein the amount of desiccant added to the container in step (b) is at least a portion of the total amount of desiccant used.

3. A method according to claim 1, wherein the nucleic acid is DNA.

4. A method according to claim 3, wherein the desiccant is present in the container during at least part of step (d).

5. A method according to claim 3, wherein the isolating step (d), which is performed within the container, comprises:
(i) grinding the material;
(ii) extracting DNA from the material with an extraction buffer; and
(iii) separating the extraction buffer and DNA from the material.

6. A method according to claim 5, wherein the desiccant is present in the container during the grinding step.

7. A method according to claim 5, wherein the desiccant is present in the container during the grinding and/or the extracting step.

8. A method according to claim 1, wherein the isolated nucleic acid is less degraded than nucleic acid that was isolated in the absence of the desiccant.

9. A method according to claim 1, wherein the desiccant is one that is inert with respect to the nucleic acid.

10. A method according to claim 9, wherein the desiccant is one of the following substances: silica, alumina, aluminum silicates, calcium sulfates, magnesium aluminum silicates, porous clay materials, diatomaceous earth, zeolites, or a salt of (poly)acrylic acid, or is a mixture comprising any of said substances.

11. A method according to claim 1, wherein the nucleic acid comprising material is of plant or animal origin.

12. A method according to claim 11, wherein the animal nucleic acid comprising material is obtained from blood, sperm, mucus muscle or skin.

13. A method according to claim 1, wherein the desiccant has a drying capacity characterized by a ratio of the desiccant to the amount of water in the nucleic acid comprising material of is at least 1.

14. A method according to claim 1, wherein the nucleic acid comprising material is dried at a temperature range selected from the group consisting of:
(a) between 0° and 50 C;
(b) between 10° and 40 C;
(c) between 15° and 30 C; and
(d) between 18° and 25 C.

15. A method according to claim 11 wherein the nucleic acid-comprising material is of human origin.

16. A method according to claim 13, wherein the ratio is greater than 2.

17. A method according to claim 3, wherein the desiccant is one that is inert with respect to the DNA.

18. A method according to claim 5, wherein the desiccant is present during at least two of steps (i)-(iii).

19. A method according to claim 5, further comprising:
(iv) transferring the extraction buffer and DNA to a second containers and
(v) optionally, separating the DNA from the extraction buffer in the second container.

20. A method according to claim 11, wherein the plant nucleic acid-comprising material is obtained from a leaf.
